(19) United States
(12) Patent Application Publication
(10) Pub. No.: US 2012/0245408 A1
(43) Pub. Date: Sep. 27, 2012

(54) METHODS AND SYSTEMS FOR IMPROVING ACTUATOR PERFORMANCE BY REDUCING TENSILE STRESSES IN PIEZOELECTRIC THIN FILMS

(75) Inventors: I-Yeu Shen, Seattle, WA (US); Cheng-Chun Lee, Chungli (TW); Clifford R. Hume, Seattle, WA (US); Guozhong Cao, Seattle, WA (US); Chuan Luo, Beijing (CN)

(73) Assignee: University of Washington through its Center for Commercialization, Seattle, WA (US)

(21) Appl. No.: 13/427,727

(22) Filed: Mar. 22, 2012

Related U.S. Application Data
(60) Provisional application No. 61/466,375, filed on Mar. 22, 2011.

(51) Int. Cl. A61F 11/04; H04R 25/00; H02N 2/06

(52) U.S. Cl. 600/25; 310/317

(57) ABSTRACT

Disclosed are methods and systems for improving actuator performance by reducing tensile stresses in piezoelectric thin films. In one embodiment, a piezoelectric actuator includes a substrate, a first electrode positioned on the substrate, a piezoelectric thin film positioned on the first electrode, and a second electrode positioned on the piezoelectric thin film. The displacement capability of the actuator is enhanced by reducing the tensile stresses of the piezoelectric thin film. In some embodiments, a constant DC voltage applied to the piezoelectric actuator generates compressive in-plane stresses, which counteract the tensile in-plane stresses. As a result, the overall tensile stresses in the actuator are reduced, and the actuator displacement is improved.

### CROSS-REFERENCE TO RELATED APPLICATION(S)

This application claims the benefit of U.S. Provisional Application 61/466,375, filed Mar. 22, 2011, which is incorporated herein by reference in its entirety.

### STATEMENT OF GOVERNMENT INTEREST

This invention was made with govenrment support under grant number CMMI-0826501 awarded by the National Science Foundation (NSF). The government has certain rights in the invention.

### TECHNICAL FIELD

The present application relates generally to piezoelectric thin films and, more specifically, to methods and systems for improving actuator performance by reducing tensile stresses in piezoelectric thin films.

### BACKGROUND

Piezoelectric thin films, such as lead zirconate titanate (PZT), have traditionally made useful actuators. Relatively small PZT actuators, however, can suffer from inherent manufacturing defects that reduce their performance. For example, in-plane tensile stresses are inherently generated in the fabrication of PZT membranes. More specifically, fabrication of PZT films generally includes the step of sintering the material at 650° C., then cooling the sintered material to room temperature. Relatively large tensile stresses are left in the PZT membrane structure during the cooling process.

When tensile in-plane stresses are present, the stiffness of the actuator increases, and the actuator displacement decreases accordingly. This limited displacement degrades the performance of the actuator. A simple way to detect the presence of high tensile stresses is to measure natural frequencies of the actuator. Often, presence of high tensile in-plane stresses is accompanied by an increase of natural frequencies. The internal stresses resulting from fabrication are difficult to control and predict. Therefore, the negative effects of the internal stresses are difficult to estimate and mitigate. Accordingly, it would be desirable to provide simple and effective techniques for reducing such manufacturing-related tensile stresses, and thereby improve actuator performance.

### BRIEF DESCRIPTION OF THE DRAWINGS

FIG. 1 is a partially schematic depiction of a piezoelectric thin film actuator configured in accordance with embodiments of the present technology.

FIGS. 2A-2F are partially schematic depictions of another piezoelectric thin film actuator at various stages of manufacture in accordance with embodiments of the present technology.

FIG. 3 is a partially schematic depiction of another piezoelectric thin film actuator configured in accordance with further embodiments of the present technology.

FIG. 4 is a partially schematic depiction of another piezoelectric thin film actuator configured in accordance with still further embodiments of the present technology.

### DETAILED DESCRIPTION

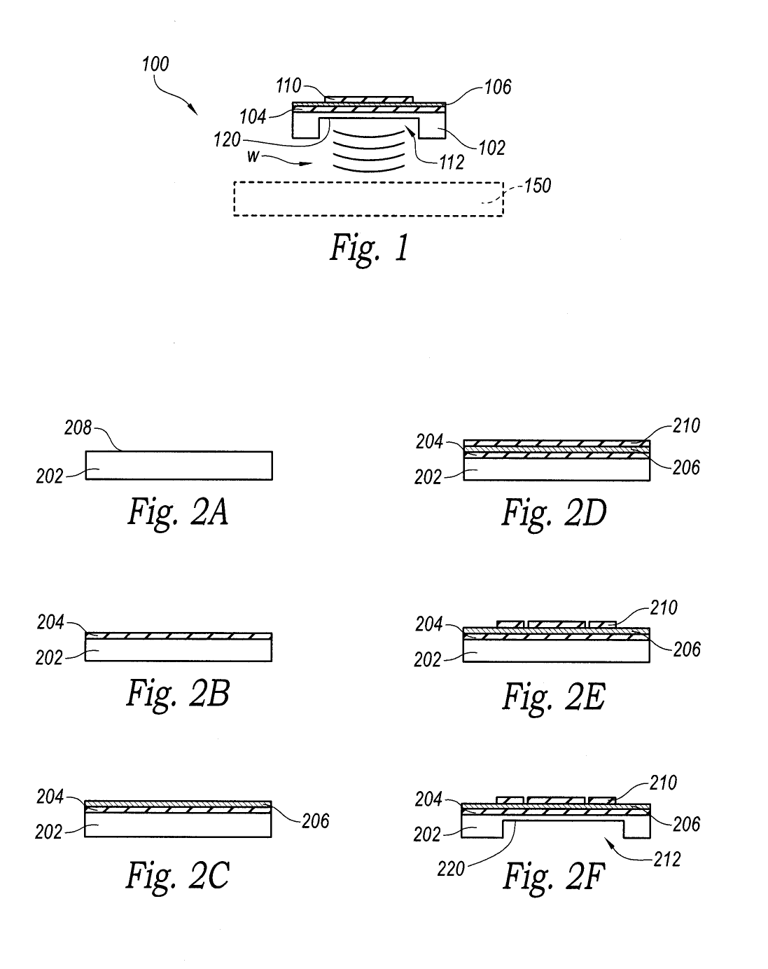

The present technology is generally related to methods and systems for improving actuator performance by reducing tensile stresses in piezoelectric thin films. In particular, specific details of several embodiments of representative piezoelectric actuators, and associated methods for enhancing actuator displacement by reducing the tensile in-plane stresses of piezoelectric membranes are described herein. Tensile in-plane stresses are a byproduct of the manufacturing process, and when such stresses are reduced or eliminated, actuator performance is improved. In one embodiment, a piezoelectric actuator includes a substrate, a first electrode positioned on the substrate, a piezoelectric thin film positioned on the first electrode, and a second electrode positioned on the piezoelectric thin film. In some embodiments of the technology, a constant DC voltage is applied to the piezoelectric actuator and generates compressive in-plane stresses, which counteract the tensile in-plane stresses created during fabrication. As a result, the overall tensile stresses in the actuator are reduced, and the actuator displacement is enhanced. A person skilled in the relevant art will understand that the present technology may have additional embodiments and that this technology may be practiced without several of the details of the embodiments described below with reference to FIGS. 1-4.

FIG. 1 is a partially schematic depiction of a piezoelectric thin film actuator 100 configured in accordance with embodiments of the present technology. The actuator 100 includes a substrate 102, a first electrode 104 coupled to the substrate 102, a PZT piezoelectric thin film 106 coupled to the first electrode 104, and a second electrode 110 coupled to the PZT thin film 106. In several embodiments, the substrate 102 comprises a silicon substrate, but other materials may be used in other embodiments. The electrodes 104 and 110 can be platinum on titanium and gold on chromium, respectively. In further embodiments, the electrodes 104, 110 can be made of other materials or combinations of materials. In some embodiments, the first electrode 104 is made of different materials than the second electrode 110, while in other embodiments the first and second electrodes 104, 110 are made of the same materials. The PZT thin film 106 may comprise alternate biocompatible piezoelectric films in other embodiments.

A portion 112 of the substrate 102 opposite the electrodes 104, 110, has been removed to expose a displaceable diaphragm suspension 120. The diaphragm suspension 120 can have various dimensions and proportions relative to the overall substrate 102. In a particular embodiment, for example, the diaphragm suspension 120 can have a surface size of 800 microns×800 microns, and a thickness of about 2.5 microns. In one embodiment, the substrate 102, the first electrode 104, the PZT thin film 106, and the second electrode 110 each have a thickness of about 1 micron. The diaphragm suspension 120 has a low structural stiffness and can be driven to displacement based, at least in part, on its relatively small thickness compared to the overall thickness of the actuator 100.

In operation, when voltage is applied between the electrodes 104, 110 across the PZT thin film 106, the contraction and extension of the PZT thin film 106 creates a bending moment to flex the diaphragm suspension 120 out of its resting plane. This vibration causes the diaphragm suspension 120 to emit pressure waves W. One exemplary application of the actuator is with intra-cochlear prostheses, in which pressure waves W emitted by the diaphragm suspension 120 can be received by a basilar membrane 150.

Acoustic stimulation from intra-cochlear prostheses requires relatively small actuators that exhibit relatively large displacements. In some embodiments, for example, to provide a 20 dBA improvement over normal hearing, an intra-cochlear acoustic actuator should provide a displacement of at least 200 nm. Conventional piezoelectric actuators generally cannot provide such a displacement, in large part due to tensile in-plane stresses in the actuator. Using the present technology, however, a constant direct current (DC) voltage (also known as a “bias voltage”) can be applied to the actuator 100 to generate compressive in-plane stresses, which counteract the tensile in-plane stresses. As a result, overall tensile stresses in the actuator 100 are reduced, and displacement of the actuator 100 is enhanced. In a particular embodiment, for example, data indicates that a bias voltage of about 5 V can lead to an approximately 30% improvement in actuator displacement.

In some embodiments, the driving signal for an intra-cochlear acoustic actuator contains a DC bias voltage component and an alternating current (AC) component driven by a power supply. In some embodiments, the AC component may not be purely sinusoidal. Instead, the AC component may contain multiple frequency components or encompass a frequency range. While the DC bias voltage reduces the tensile in-plane stresses, allowing the actuator to achieve larger displacements, the AC voltage component delivers the acoustic signal required to rehabilitate the patient's hearing loss. In yet another embodiment, a DC bias is used to tune the performance of the piezoelectric actuator 100 to a predefined standard. Since in-plane stresses are difficult to control in fabrication, they can vary significantly from one actuator to another. In this embodiment, the DC bias voltage can be used as a way to adjust individual actuators (such as the actuator 100) to achieve a predefined displacement standard such that product variations can be minimized. This feature is expected to substantially reduce the need to maintain strict processing controls during fabrication of the piezoelectric actuators 100, and thereby significantly reduce fabrication costs and increase throughput.

In addition to intra-cochlear prostheses, the thin film actuator 100 may be applied to numerous other applications, such as nozzles, micro scanners, micro-deformable mirrors, energy harvesters, micro pumps, micro high-fidelity speakers, micro protein desportion devices, fuel cell membranes, micro energy generators, micro mass sensing devices, ultrasonic transducers, acoustic transducers, micropressure sensors, and intra-vestibular prostheses. For many of these applications, it is desirable to maximize the actuator constant, which is defined as the diaphragm suspension 120 displacement generated per unit voltage applied. For example, as mentioned above, microactuators used to generate acoustic pressure waves in intra-cochlear applications may have a specification of 200 nm of displacement. If the actuator constant is large, only a small voltage is needed to drive the actuator 100 to achieve the desired displacement.

FIGS. 2A-2F are partially schematic illustrations of a piezoelectric thin film actuator at various stages of manufacture in accordance with an embodiment of the technology. The actuator described below with reference to FIGS. 2A-2F includes a number of features similar to the actuator 100 of FIG. 1. Referring first to FIG. 2A, a first surface 208 of a substrate 202 is oxidized in a furnace to grow a SiO₂ layer. In a particular embodiment, the substrate 202 is oxidized at 1045° C. for approximately two hours to grow a SiO₂ layer of 500 nm thick on the surface 208. A layer of silicon nitride (not shown) is then deposited on the surface 208. In a particular embodiment, the silicon nitride is 200 nm thick and is deposited by plasma enhanced chemical vapor deposition (PECVD). In other embodiments, however, the silicon nitride may have a different thickness and/or be deposited using other suitable techniques.

As shown in FIG. 2B, a first electrode 204 is then positioned on the treated surface 208 of the substrate 202. In one embodiment, the first electrode 204 can comprise platinum and titanium layers with thicknesses of 100 nm and 50 nm, respectively. FIG. 2C illustrates the substrate 202 after the addition of a PZT thin film 206. In some embodiments, the PZT thin film is spin-coated three times. In a particular embodiment, for the first two coatings, the sintering temperature is 650° C. for 15 minutes. For the third coating, the PZT is diluted by 50% by acetic acid and the sintering temperature is 450° C. for 10 minutes.

FIG. 2D illustrates the substrate 202 after a second electrode 210 has been added to the PZT thin film 206. In some embodiments, the second electrode 210 can comprise layers of gold and chromium deposited through evaporation. In the embodiment illustrated in FIG. 2E, the second electrode 210 can be patterned by removing portions of the electrode 210 by a lift-off technique. In other embodiments, the second electrode 210 can be patterned by removing portion of the electrode 210 via etching or other techniques. Finally, as shown in FIG. 2F, a portion 212 of the substrate 202 is removed opposite the electrodes 204, 210. In some embodiments, the removed portion 212 is eliminated by an etching process (e.g., by deep-reactive ion etch) to expose a diaphragm suspension 220. In further embodiments, the fabricated actuator may be packaged via a thin layer of coating (e.g., parylene), forming a temporary or hermetic seal that allows the fabricated actuator to be used in aqueous environments.

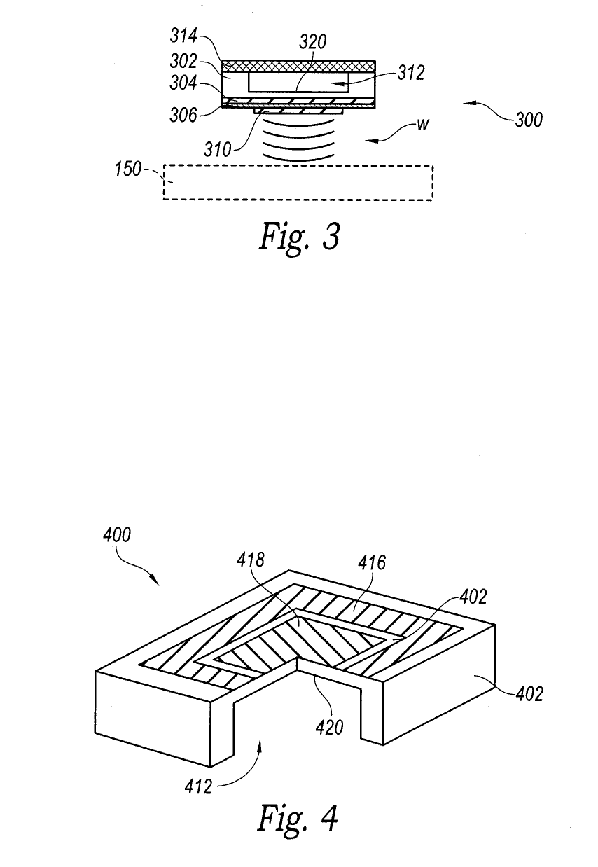

FIG. 3 is a partially schematic depiction of another piezoelectric thin film actuator 300 configured in accordance with further embodiments of the present technology. The actuator 300 includes several features generally similar to those discussed above with reference to FIGS. 1-2F. For example, the actuator 300 includes a substrate 302, a first electrode 304, a PZT thin film 306, a second electrode 310, and a diaphragm suspension 320 positioned adjacent to a removed portion 312 of the substrate 302.

The actuator 300 further includes a buffer 314 coupled to the substrate 302 and enclosing the removed portion 312. The buffer 314 can comprise a thick glass or silicon (e.g., polydimethylsiloxane) buffer to seal the cavity. As the PZT thin film 306 deflects, the volume of the actuator 300 changes, thus further exciting the basilar membrane 150. The buffer 314 can prevent fluid between the diaphragm suspension 320 and the basilar membrane 150 from circulating around the diaphragm suspension 320.

FIG. 4 is a partially schematic depiction of another piezoelectric thin film actuator 400 configured in accordance with still further embodiments of the present technology. The actuator 400 includes several features similar to those discussed above with reference to FIGS. 1-3. In this embodiment, however, instead of a single “top” electrode, the actuator 400 includes two “top” electrodes 416, 418 positioned on the center and periphery of an outer surface of a substrate 402. In the illustrated embodiment, the electrodes 416, 418 are concentric, but may have other arrangements in other embodiments. In several embodiments, the electrodes 416, 418 coincide with a suspended diaphragm 420 over a removed portion 412. In other words, the electrodes 416, 418 vertically overlap the entire diaphragm suspension 420.

In some embodiments, the two electrodes 416, 418 can be driven with out-of-phase voltage, enhancing displacement of the diaphragm suspension 420. More specifically, since the diaphragm suspension 420 is fixed at its four edges, the overall strain inside the diaphragm suspension 420 must be zero. If the center electrode 418 has a positive strain, the peripheral electrode 416 must have a negative strain to maintain the zero average strain. Therefore, driving the electrodes 416, 418 with independent, out of phase voltage (e.g., 180° out of phase) will enhance the displacement of the entire diaphragm suspension 420. The degree to which the voltages are out of phase can vary. In some embodiments, for example, the voltages are out of phase between about 90° and about 180°. When large internal stresses are present warping the actuator, in-phase voltage (i.e., 0° out of phase) applied to electrodes 416, 418 can enhance the actuator displacement. In some embodiments, both the center and peripheral electrodes 416, 418 are driven by the AC component, while in other embodiments one electrode is driven by a DC component while the other is driven by the AC component. In a still further embodiment, both electrodes 416, 418 are driven by DC components, while one electrode is superimposed with the AC component.

From the foregoing, it will be appreciated that specific embodiments of the technology have been described herein for purposes of illustration, but that various modifications may be made without deviating from the disclosure. Additionally, material choices for the actuator components and substrates can vary in different embodiments of the disclosure. In certain embodiments, the actuators can be used in applications other than those described above. Furthermore, the term substrate refers to supports for individual actuators and larger wafers or workpieces upon which a plurality of actuators are formed or mounted. Several of the figures shown and discussed herein are not to scale. Certain elements of one embodiment may be combined with other embodiments, in addition to or in lieu of the elements of the other embodiments, and a given structure can include multiple of the same element. Accordingly, the disclosure can encompass other embodiments not expressly shown or described herein.

I/We claim:

1. A method for improving a displacement performance of a piezoelectric actuator, the method comprising:
providing an actuator including—
a substrate having a displaceable diaphragm suspension portion;
a piezoelectric thin film coupled to the substrate;
a first electrode on the piezoelectric thin; and
a second electrode on the piezoelectric thin film;
driving the piezoelectric actuator with an AC driving signal component; and
driving the piezoelectric actuator with a DC bias signal component.

2. The method of claim 1 wherein driving the piezoelectric actuator with a DC bias signal component comprises driving the piezoelectric actuator with a DC bias signal component of about +/−5 V.

3. The method of claim 1 wherein driving the piezoelectric actuator with a DC bias signal component comprises driving the piezoelectric actuator with a DC bias signal component having a strength sufficient to tune a performance characteristic of the piezoelectric actuator to a predefined standard.

4. The method of claim 1 wherein improving a displacement performance of a piezoelectric actuator comprises improving a displacement performance of a piezoelectric actuator in an intra-cochlear prosthesis for a hearing impaired patient, and wherein:
driving the piezoelectric actuator with an AC driving signal component comprises generating an acoustic signal in the patient's cochlea; and
driving the piezoelectric actuator with a DC bias signal component comprises reducing tensile stresses within the piezoelectric thin film.

5. The method of claim 1 wherein the piezoelectric thin film comprises lead zirconate titanate.

6. The method of claim 1 wherein the piezoelectric thin film comprises a biocompatible material.

7. The method of claim 1, further comprising packaging the actuator with a hermetic seal.

8. The method of claim 1 wherein the actuator further includes a buffer coupled to the substrate, and wherein the buffer, along with the substrate, encloses an open area adjacent to the diaphragm suspension portion.

9. An intra-cochlear prosthesis for a hearing impaired patient, the intra-cochlear prosthesis comprising:
a power supply;
a piezoelectric actuator operably coupled to the power supply; and
a processor for controlling the piezoelectric actuator, the processor configured to implement instructions for
generating an acoustic signal in the patient's cochlea with a first driving signal; and
reducing tensile stresses within the piezoelectric actuator with a second driving signal.

10. The intra-cochlear prosthesis of claim 9 wherein the first driving signal is an AC driving signal and the second driving signal is a DC bias signal.

11. The intra-cochlear prosthesis of claim 9 wherein the second driving signal is a DC bias signal of about +/−5 V.

12. The intra-cochlear prosthesis of claim 9, further comprising a plurality of stimulation electrodes logically coupled to the processor and operably coupled to the power supply.

13. The intra-cochlear prosthesis of claim 9 wherein the piezoelectric actuator comprises a first stimulation electrode positioned on a substrate and a second stimulation electrode positioned on the substrate and concentrically surrounding the first stimulation electrode.

14. The intra-cochlear prosthesis of claim 9 wherein the processor is configured to implement instructions for (a) generating an acoustic signal in the patient's cochlea with a first AC driving signal and (b) enhancing displacement of the piezoelectric actuator with a second AC driving signal out of phase with the first AC driving signal.

15. The intra-cochlear prosthesis of claim 14 wherein a phase difference between the first and the second AC driving signals is selected to maximize the displacement of the piezoelectric actuator.

16. The intra-cochlear prosthesis of claim 9 wherein the processor is configured to implement instructions for reducing tensile stresses within the piezoelectric actuator with an AC driving signal superimposed with a DC signal.

17. The intra-cochlear prosthesis of claim 9 wherein the piezoelectric actuator comprises:
a substrate having a portion of reduced thickness comprising a diaphragm suspension;
a first electrode on the substrate;
a piezoelectric thin film on the first electrode; and
a second electrode on the piezoelectric thin film, wherein the first electrode and the second electrode are in operable communication with the power supply.

18. The intra-cochlear prosthesis of claim 17 wherein the piezoelectric actuator further comprises a buffer coupled to the substrate opposite the first electrode, wherein the buffer, in conjunction with the substrate, encloses a substrate cavity adjacent to the diaphragm suspension.

19. The intra-cochlear prosthesis of claim 9 wherein the first driving signal is an AC driving signal and the second driving signal is a DC bias signal sufficient to tune a performance characteristic of the piezoelectric actuator to a predefined standard.

20. The intra-cochlear prosthesis of claim 9, further comprising a second piezoelectric actuator.

21. A piezoelectric actuator system, comprising:
a power supply;
a piezoelectric actuator operably coupled to the power supply and configured to generate a displacing force ,wherein the piezoelectric actuator comprises
a substrate having a portion of reduced thickness comprising a diaphragm suspension;
a piezoelectric thin film coupled to the substrate;
a first electrode on the piezoelectric thin; and
a second electrode on the piezoelectric thin film, wherein the first electrode and second electrode are in operable communication with the power supply; and
a processor for controlling the piezoelectric actuator, wherein the processor is configured to implement instructions for driving the piezoelectric actuator with a driving signal comprising a DC bias component, wherein the DC bias component enables the piezoelectric actuator to generate a greater displacement than would be possible without the DC bias component.

22. The system of claim 21 wherein the processor is configured to implement instructions for driving the piezoelectric actuator with a driving signal comprising a DC bias component of about +/−5 V.

23. The system of claim 21 wherein the processor is further configured to implement instructions for driving the piezoelectric actuator with an AC driving component.
